(19) United States
(12) Patent Application Publication
(10) Pub. No.: US 2003/0175821 A1
(43) Pub. Date: Sep. 18, 2003

(54) Apparatus and method for microscopic comet assay

(76) Inventors: Rex A. Hoover, Sumerduck, VA (US); Robert G. Hoover, Sumerduck, VA (US)

Correspondence Address: Chandrakant C. Shroff, 600 North Pickett Street, Alexandria, VA, 22304-2110, US

(21) Appl. No.: 10/331,321

(22) Filed: Dec. 31, 2002

Related U.S. Application Data
(60) Provisional application No. 60/363,903, filed on Mar. 14, 2002.

(51) Int. Cl. G01N 33/53; G01N 33/567; G06K 9/00; C12M 1/34

(52) U.S. Cl. 435/7.2; 435/287.2; 382/128

(57) ABSTRACT

An automated cometary assay system is disclosed wherein light filtered to the excitation frequency of a fluorescing compound is directed onto the sample. A second filter isolates the emission frequency and the resulting image is directed at a low-light camera.

### BACKGROUND OF THE INVENTION

1. Field of the Invention

The present invention relates generally to microscopy. More specifically, it relates to epifluorescence and the measurement of comet halos. Even more specifically, it relates to the automated measurement of the length of comet trails created by broken DNA strands under electrophoresis. The system includes a light source, lenses, a filter, mirrors, and a low-light sensitive camera.

2. Description of the Prior Art

One of the ways to measure the toxicity of an agent in relation to certain tissue types is by means of a “comet assay”. In short, the cells are exposed to the agent in question and are then embedded in a gel and exposed to a current: i.e. electrophoresis. After a predetermined time, the cells are placed on a slide and are stained with a fluorescing material. The broken DNA strands have migrated out of the cell an under certain frequencies of light the “halo” that is created by the migrating strands can be seen and measured. At present this measurement has been done manually, and the present apparatus that are used in the test create large amounts of heat due to the intensity of the light required. The samples that are being measured thus degrade at a fast rate, adding to the pressure placed on the technician or researcher attempting to determine the level of damage in the nucleus. The present invention, due to its novel construction, prevents this rapid sample degeneration and allows the user to see the halos or trails for up to an hour with accuracy before the fluorescence starts to fade. Additionally, the low-level light source, the light filter, and the low-light camera of the present invention do not require a light intensity that creates the amount of heat that so rapidly degrades the sample as is common in the present art devices. Additionally, the present invention includes an automated algorithm and system that allows the user to place the samples to be tested in a light sealed box, and then allow the instant invention to do the assay independently in less than half an hour. Thus, the toxicity of various materials can be rapidly determined in relation to a variety of tissue types. For example, liver cells could be exposed to various additives that would pass the stomach lining and be present in the bloodstream after ingestion.

During a search at the U.S. Patent and Trademark Office, a number of relevant patents were uncovered and they will be discussed below.

In U.S. Pat. No. 4,695,548 issued to Charles R. Cantor et al. On Sep. 22, 1987 there are disclosed gel inserts for electrophoresis. This is unlike the present invention in that there are no optical components disclosed.

U.S. Pat. No. 4,870,004 issued to Thomas J. Conroy et al. On Sep. 26, 1989 discloses an apparatus and method of analyzing nucleic acids. This involves electrophoresis however radiation detectors are used to determine when specific groups pass a predetermined point. This is clearly dissimilar from the present invention.

In U.S. Pat. No. 5,852,4989 issued to Douglas C. Youvan et al. On Dec. 22, 1998 an optical instrument with a filter is disclosed. This is unlike the present invention in that no measuring of comet halos is discussed.

In U.S. Pat. No. 5,989,835 issued to R. Terry Dunlay et al. A system for cell based screening is disclosed. Fluorescing reporter molecules are provided and an optical system reports the activity thereof. Unlike the present invention, electrophoresis is not used.

Lastly, in U.S. Pat. No. 6,154,282 issued to Lothar Lilge et al. On Nov. 28, 200 discloses an illuminator for fluorescent or phosphorescent microscopy. Again, no electrophoresis to separate broke DNA fragments is disclosed.

Thus, while the foregoing overview of prior art indicates it to be well known to use the measurement of comet halos to ascertain the damage done to DNA in a certain tissue type, none of the inventions discussed above, either alone or in combination, describe the instant invention as claimed.

### SUMMARY OF THE INVENTION

An automated comet assay system wherein light filtered to the excitation frequency of a fluorescing compound is directed onto the sample. A second filter isolates the emission frequency and the resulting image is directed at a low-light camera.

To achieve the foregoing and other advantages, the present invention, briefly described, provides an apparatus and method for microscopic comet assay that overcome the disadvantages of the prior art.

Thus it is a principal object of the invention to provide an apparatus and method for microscopic comet assay that is completely automated.

It is a further object of the invention to provide an apparatus and method for microscopic comet assay wherein low levels of light are used to prevent degradation of the sample and to allow a longer viewing time.

Still yet a further object of the invention is to provide an apparatus and method for microscopic comet assay wherein a sensitive CCD image sensor is used to transmit the comet trail to a computer.

Yet another object of the invention is to provide an apparatus and method for microscopic comet assay wherein a light filter is used to maximize the epifluorescence of the stained DNA fragments.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawings wherein:

FIG. 1 is a perspective view of the present invention showing the sample stage, light source, lenses, mirrors, filters, and camera.

FIG. 2 is a diagrammatic view of the optical components of the invention.

FIG. 3 is a graph showing the excitation and emission wavelengths of the epifluorescing components

FIG. 4 is a graph showing the transmission percentage of the filter set used in the instant invention.

### DESCRIPTION OF THE PREFERRED EMBODIMENTS

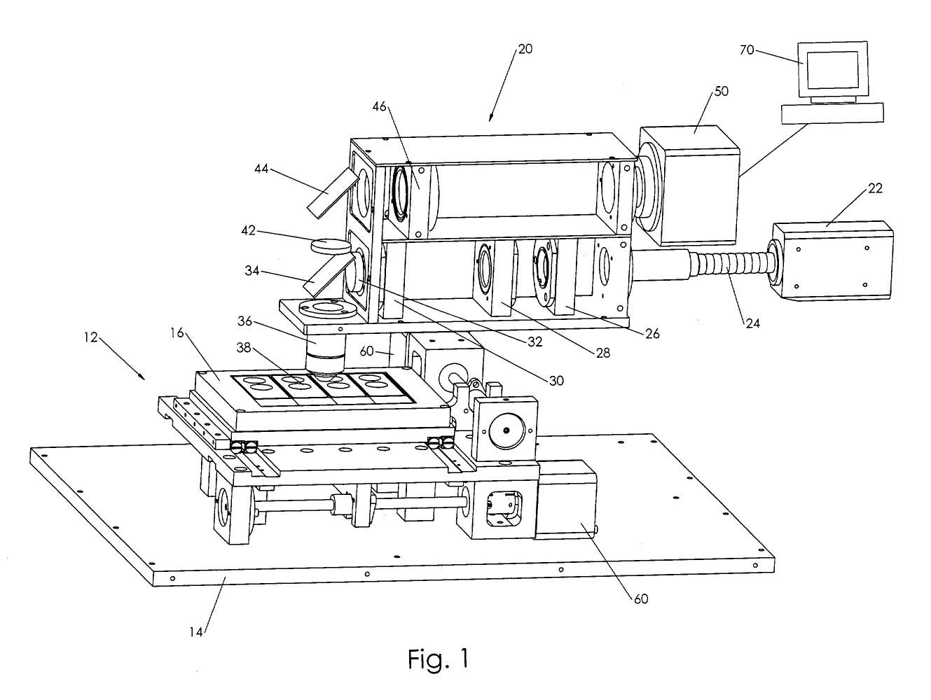

Referring first to FIG. 1, the instant invention is indicated generally at 10. It should be understood that the stage and lens portion (discussed below) are contained within a light-tight box (not shown).

The sample stage portion 12 is mounted on a base 14. The sample stage base 14 includes a slide holding area 16 that, in the embodiment described herein, holds two slides. It should be emphasized that fewer or more slides could be held in the holding area 16. Placed in these slides are cells that have been exposed to an environment that the researcher wishes to study. This could be exposure to a chemical, electromagnetic radiation, or the like. The cells which are placed in the environment are also chosen by the user: for example liver cells could be exposed to chemicals that would be present in the bloodstream of a person who had ingested an experimental drug. After a set period of exposure time, the cells are placed into an electrophoresis column or gel. Voltage is applied across the gel and the broken or fragmented strands of DNA (if any such exist) resulting from the environmental exposure will migrate from the nucleus of the cell. The cells are then placed on the slides and stained with a compound that will bind to the DNA and epifluoresce under certain frequencies of light.

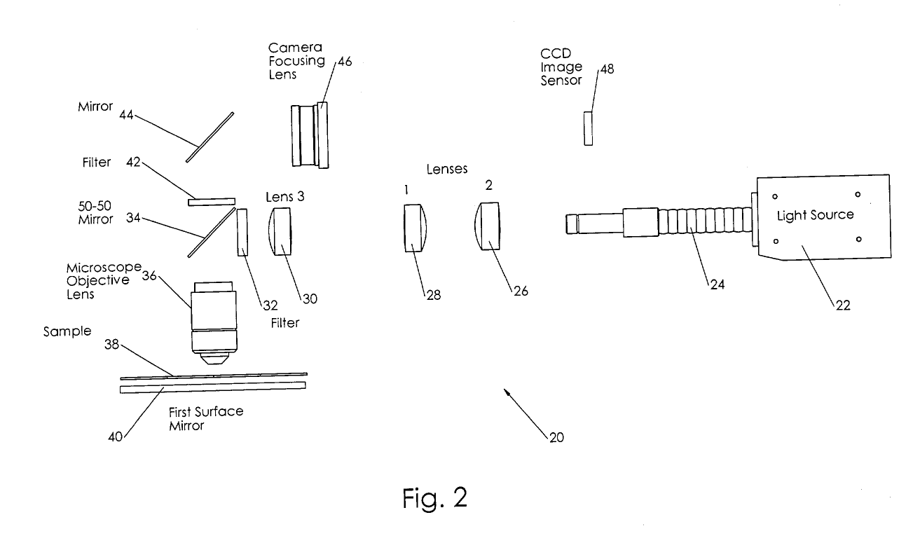

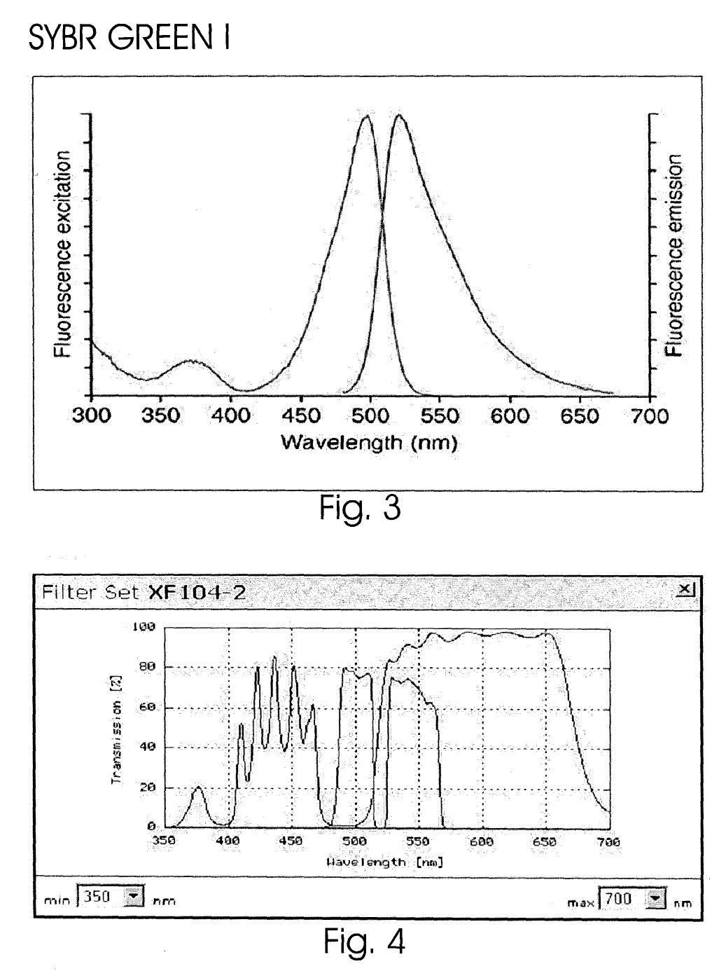

The discussion now turns to the optical portion 20 of the present invention. This is best seen while referring to both FIGS. 1 and 2. The source of illumination is a light box 22 that is connected to the light-tight area (not shown) by a fiber optic conduit 24. The light passes through a pair of lenses 26, 28 as indicated by arrow A1, then the lens 30 as shown in arrow A2. Next, the light passes through a filter 32 (which the transmission percentage is seen in FIG. 4), and then is reflected off a 50-50 mirror 34 through the microscope's objective lens 36. In one embodiment of the invention, the sample 38 to be studied is located above a first surface mirror 40 that reflects the light even further back through the sample. The light from the epifluorescence passes back through the 50-50 mirror 34, through another filter 42 that blocks out the original light frequency (see FIG. 3) and then is reflected back off another first surface mirror 44, into the camera focusing lens 46 and into the CCD image sensor 48 of the low-light camera 50. Low-light camera 50 is a type used for security work where only a very small amount of light is necessary for the image to register. This feature allows the amount of light used overall in the instant invention to be very small, which cuts down on the heat produced during the imaging process, and the subsequent degradation of the sample. It has been noted by the inventor that in the common art devices used to measure the comet halos, the light used is so intense that the samples begin to degrade and the halos cease to be visible after only one minute or so. With the present invention, the halos are still visible after twenty minutes, albeit more dimly than originally after staining.

Note that because of the second filter 42, that blocks out the excitation wavelength and passes the emission wavelength (again, see FIG. 3) adequate signal to noise ratios are achieved with relatively low intensities of light.

Referring back to FIG. 1, the stage of the unit is movable in that two stepper motors 60 running worm gears move the stage as desired by the computer 70. This allows for the automated assay to take place as follows:

A typical assay is comprised of 20 sample areas contained on two slides each with two 14 mm diameter sample areas thereon. These areas are scanned until a total of approximately 1000 cells are located and measured. It has been observed with this system that each field of view will contain cells numbering up to 20 or more and that each field of view will take about five seconds to locate, measure, And define the geometry of the cell. The head area of the cell is calculated and then subtracted from the total cell area. The length is measured from the edge of the head to the furthermost end of the tail area. Then the tail moment is calculated by multiplying each tail pixel intensity by the distance from the edge of the cell head divided by the total head and tail intensity added together.

It should be emphasized that the instant invention is not in any way limited to the embodiments as they are described above but encompasses all embodiments as described in the scope of then following claims.

What is claimed as being new and desired to be protected by Letters Patent of the United States is as follows:

1. An apparatus for microscopic comet assay comprising:
a light-tight box;
a stage located within said light-tight box adapted to hold at least one slide;
stage movement motors located within said light-tight box for moving said stage in a horizontal plane;
a light source;
a first filter within said light-tight box for filtering the light from said light source to a first, predetermined excitation frequency;
a first light direction means within said light-tight box for directing the light at said excitation frequency to said at least one slide located on said stage;
a second filter within said light-tight box for filtering reflected light from said slide to a second, predetermined emission frequency;
a second light direction means within said light-tight box for directing the light at said emission frequency to a CCD image sensor; whereby
cells that are stained with a fluorescing compound may be placed on said at least one slide located on said stage and are illuminated with light at said excitation frequency adapted to react with said fluorescing compound and where the light then generated by said fluorescing compound is further filtered through said second filter such that only light at said emission frequency of said fluorescing compound is passed to said CCD image sensor.

2. The apparatus as claimed in claim 1, wherein a first set of lenses is placed between said light source and said first filter.

3. The apparatus according to claim 1 wherein a second set of lenses is located between said second filter and said CCD image sensor.

4. The apparatus according to claim 1, wherein said first filter passes light between 425 and 575 nm.

5. The apparatus according to claim 4 wherein said second filter passes light between 475 and 625 nm.

6. The apparatus according to claim 1, wherein said first light direction means comprises a 50-50 mirror.

7. The apparatus according to claim 6 wherein said second light direction means comprises a first surface mirror.

8. A method of microscopic comet assay comprising the steps of:
providing a light-tight box environment;
exposing a predetermined cell tissue to a predetermined environment;
submitting said exposed cells to electrophoresis;
staining said cell tissue with a fluorescing compound;
directing light filtered to an excitation frequency of said fluorescing compound onto said cell tissue;
magnifying the resulting epifluorescence;
filtering said resulting epifluorescence to the emission frequency of said fluorescing compound; and
directing said light filtered to said emission frequency to a CCD image sensor.

9. The method according to claim 8 further comprising the steps of:
determining the total area of the cell image in said CCD image sensor;
determining the head area of the cell image in said CCD image sensor;
subtracting said head area from said total area to arrive at the tail area of said cell image
measuring the head intensity and the tail intensity to define an intensity percentage;
measuring each pixel in said tail area and multiplying by the distance from an edge of said tail area, then dividing by the total of said head intensity and said tail intensity to arrive at a tail moment value.
